(19) United States
(12) Patent Application Publication
(10) Pub. No.: US 2019/0206550 A1
(43) Pub. Date: Jul. 4, 2019

(54) METHOD FOR MANAGING ADMISSION OF PATIENTS INTO INTENSIVE CARE UNITS IN A HOSPITAL NETWORK

(71) Applicant: City University of Hong Kong, Kowloon (HK)

(72) Inventors: Wing Ming Eric Wong, Kowloon (HK); Yin Chi Chan, Kowloon (HK); Gavin Joynt, Shatin (HK); Moshe Zukerman, Kowloon (HK)

(21) Appl. No.: 15/859,876

(22) Filed: Jan. 2, 2018

(51) Int. Cl. G16H 40/20; G16H 50/20; G06N 7/00; G06F 17/10

(52) U.S. Cl. CPC G16H 40/20; G06F 17/10; G06N 7/005; G16H 50/20

(57) ABSTRACT

A method for managing admission of patients into one of a plurality of intensive care units in a hospital network includes classifying a patient arriving at a first intensive care unit as one of an external emergency patient, an internal emergency patient, and an elective patient; and determining whether to admit the patient to the intensive care units in the hospital network based on the classification. An external emergency patient is admitted to the first intensive care unit if a vacancy in the first intensive care unit is above a first threshold. An internal emergency patient is admitted to the first intensive care unit if the first intensive care unit has vacancy. An elective patient is admitted to the first intensive care unit if a vacancy in the first intensive care unit is above a second threshold. The first and second thresholds are independent of patient classification.

### TECHNICAL FIELD

The invention relates to a method for managing admission of patients into intensive care units in a hospital network.

### BACKGROUND

Intensive care unit (ICU) in a hospital is a crucial and expensive resource, as an ICU bed place can cost up to six times the cost of a regular hospital bed place. As a result, ICUs are frequently under-resourced and over-utilized. In addition, congestion in the ICU has a knock-on effect on the rest of the hospital system, for example in the form of deferred elective operations, and can lead to increased rates of death or ICU re-admission (due to early discharge from the ICU). Therefore, efficient management of such units is important.

### SUMMARY OF THE INVENTION

In accordance with a first aspect of the invention, there is provided a method for managing admission of a patient into one of a plurality of intensive care units in a hospital network, comprising: classifying a patient arriving at a first intensive care unit of the plurality of intensive care units as one of an external emergency patient, an internal emergency patient, and an elective patient; and determining whether to admit the patient to the intensive care units in the hospital network based on the classification so as to admit an external emergency patient to the first intensive care unit if a vacancy in the first intensive care unit exists and is above a first threshold; admit an internal emergency patient to the first intensive care unit if the first intensive care unit has vacancy; and admit an elective patient to the first intensive care unit if a vacancy in the first intensive care unit exists and is above a second threshold; wherein the first and second thresholds are independent of the classification of the patient.

Preferably, the method further comprises rejecting admission of the elective patient to the intensive care units in the hospital network if vacancy at the first intensive care unit falls below the second threshold.

Preferably, the method further comprises rejecting admission of the external emergency patient to the first intensive care unit if a vacancy in the first intensive care unit exists and is below a first threshold; and determining whether to admit the external emergency patient to another intensive care unit in the hospital network.

Preferably, the determination of whether to admit the external emergency patient to another intensive care unit in the hospital network is based on an overflow control method.

Preferably, the overflow control method comprises repeating the determination step for each of the other intensive care units in a hospital network.

Preferably, the method further comprises admitting the external emergency patient to another intensive care unit in the hospital network if a vacancy in the respective intensive care unit exists and is above a respective third threshold.

Preferably, the method further comprises rejecting admission of the external emergency patient to the intensive care units in the hospital network if vacancies at all of the respective intensive care units are below the first threshold or the respective third threshold.

Preferably, the first and third thresholds are equal.

Preferably, the first and third thresholds are adjustable dynamically. Alternatively, the first and third thresholds are fixed.

Preferably, the first and second thresholds are adjustable dynamically. Alternatively, the first and second thresholds are fixed.

Preferably, the method further comprises evaluating a first quality of service for internal emergency patients and a second quality of service for elective patients based on an exponential decomposition method with moment matching.

Preferably, the method further comprises evaluating a third quality of service for external emergency patients based on an information exchange surrogate approximation method.

Preferably, the method further comprises evaluating a first quality of service for internal emergency patients and a second quality of service for elective patients based on an exponential decomposition method with moment matching; evaluating a third quality of service for external emergency patients based on an information exchange surrogate approximation method; and determining the first, second and third thresholds based on the first, second, and third evaluated quality of service.

In accordance with a second aspect of the invention, there is provided system for managing admission of a patient into one of a plurality of intensive care units in a hospital network, comprising: means for classifying a patient arriving at a first intensive care unit of the plurality of intensive care units as one of an external emergency patient, an internal emergency patient, and an elective patient; and means for determining whether to admit the patient to the intensive care units in the hospital network based on the classification so as to admit an external emergency patient to the first intensive care unit if a vacancy in the first intensive care unit exists and is above a first threshold; admit an internal emergency patient to the first intensive care unit if the first intensive care unit has vacancy; and admit an elective patient to the first intensive care unit if a vacancy in the first intensive care unit exists and is above a second threshold; wherein the first and second thresholds are independent of the classification of the patient.

Preferably, the system further comprises various means for performing the method of the first aspect.

In accordance with a third aspect of the invention, there is provided a non-transitory computer readable medium for storing computer instructions that, when executed by one or more processors, causes the one or more processors to perform the method of the first aspect.

### BRIEF DESCRIPTION OF THE DRAWINGS

Embodiments of the invention will now be described, by way of example, with reference to the accompanying drawings in which:

FIG. 1 is a schematic diagram showing an exemplary ICU network with two ICUs (wherein solid arrows, dashed arrows, and dotted arrows represent external emergency, internal emergency, and elective patients, respectively) in accordance with one embodiment of the invention;

FIG. 2 is a schematic diagram illustrating a virtual ICU policy (wherein solid arrows, dashed arrows, and dotted arrows represent external emergency, internal emergency, and elective patients, respectively);

FIG. 3 is a schematic diagram illustrating a threshold policy in one embodiment of the invention (wherein solid arrows, dashed arrows, and dotted arrows represent external emergency, internal emergency, and elective patients, respectively);

FIG. 4A is a graph showing a simulated result of the overall blocking probability of external emergency patients (B) in an ICU network of three ICUs with respect to increases in the offered load;

FIG. 4B is a graph showing a simulated result of the mean number of temporary overbeds for internal emergency patients (T) in an ICU network of three ICUs with respect to increases in the offered load;

FIG. 4C is a graph showing a simulated result the of overall deferral probability of elective patients (D) in an ICU network of three ICUs with respect to increases in the offered load;

FIG. 5 is a graph showing the sensitivity of the overall blocking probability of external emergency patients (B), the mean number of temporary overbeds for internal emergency patients (T), and the overall deferral probability of elective patients to the patient length of stay distribution (D) (the superscript represents the variance of a lognormal patient length-of-stay distribution; no superscript represents an exponential patient length-of-stay distribution);

FIG. 6A is a graph showing relative errors for the overall blocking probability of external emergency patients (B) for Exponential Decomposition (ED) and Information Exchange Surrogate Approximation (IESA);

FIG. 6B is a graph showing relative errors for the mean number of temporary overbeds for internal emergency patients (T) for Exponential Decomposition (ED) and Information Exchange Surrogate Approximation (IESA);

FIG. 6C is a graph showing relative errors for the overall deferral probability of elective patients (D) for Exponential Decomposition (ED) and Information Exchange Surrogate Approximation (IESA);

FIG. 7A is a graph showing relative errors for the mean number of temporary overbeds for internal emergency patients (T) for Exponential Decomposition incorporating moment-matching (EDm);

FIG. 7B is a graph showing relative errors for the overall deferral probability of elective patients (D) for Exponential Decomposition incorporating moment-matching (EDm); and

FIG. 8 is a schematic diagram of an information handling system that can be configured to operate the method in one embodiment of the invention.

### DETAILED DESCRIPTION OF THE PREFERRED EMBODIMENT

### 1 Introduction

Despite evidence that cooperation between multiple ICUs in a region can reduce the rejection rate of intensive care patients, many hospital regions currently have no strategy for doing so. Instead, most ICUs currently seek transfer of overflow patients on an ad-hoc basis, without centralized systems or systematic polices (such as the one proposed in the present embodiment of the invention) to coordinate capacity and utilization issues. Therefore, the practical behavior of ICUs can and should change for the better. The current lack of centralized systems for ICU coordination may be because places sophisticated enough to introduce such systems are generally well-resourced. However, shortages are predicted in the future due to increasing costs and aging populations, even in well-resourced countries. Systems like this are thus likely to be needed. Furthermore, cooperation between ICUs to improve resource use may prove vital during periods of unexpected increases in ICU demand, such as an outbreak of acute infections.

### 1.1 Classification of ICU Patients

In one embodiment of the invention, there is provided an analytical model for a network of ICUs in which patients are classified into three types: external emergency patients, internal emergency patients, and elective patients. External emergency patients are those arriving from ambulatory care (including air transport). Internal emergency patients arrive to an ICU from other departments in the same hospital. Elective patients correspond to patients with planned operations requiring post-operative ICU stay.

The inventors of the present invention have devised, through research, that due to legal, logistical, and economic concerns regarding patient admission and transfer, efficient resource planning and daily operation of ICU networks, subject to meeting all quality-of-service (QoS) constraints, can form a major challenge. In many cases, internal emergency and elective patients must be served at the ICU of the hospital from which they originate. Furthermore, for economic reasons, ICUs may reject external emergency patients in favor of elective patients awaiting planned operations. On account of the different requirements for different ICU patient types, and of the inequities in patient outcomes caused by current policies, any new policy for the admission of patients to an ICU network must take fairness into account in addition to the overall patient rejection rate. This may require restricting ICU services to patients most likely to benefit. In fact, maximizing the number of beds available to each patient without reservation does not even necessarily lead to the lowest overall patient rejection rate, as demonstrated below in Section 3.4.3.

### 1.2 Cooperation Between Multiple Medical Units in a Region

An existing method for improving the QoS of hospital patients is the pooling of resources from multiple hospitals within a region. An example of inter-hospital cooperation is given in McManus M L, Long M C, Cooper A, Litvak E (2004) Queuing theory accurately models the need for critical care resources. Anesthesiology 100(5):1271-1276, which describes an ICU in the United States where “external requests for transfer” of a patient to an ICU may be “diverted to other institutions in the region” during times of congestion. This corresponds to the concept of overflow for external emergency patients in the analytical model of Litvak N, van Rijsbergen M, Boucherie RJ, van Houdenhoven M (2008) Managing the overflow of intensive care patients. European Journal of Operational Research 185(3)998-1010 (referred to as “Litvak” below). Additionally, overflow of internal emergency patients is “accommodated in off-service care sites” such as a post-anesthesia care unit or a separate, specialized cardiac ICU.

### 1.3 Analytical Approximation Methods for the Performance Evaluation and Resource Planning of ICU Networks

To illustrate the need for approximate QoS evaluation, it is noted that the number of system states in an ICU network, under both the virtual ICU model and the proposed model, is exponential in the number of ICUs in the network.

The method in the present embodiment combines two existing analytical approximation methods, namely exponential decomposition (ED), also known as the Erlang fixed-point approximation, and the Information Exchange Surrogate Approximation (IESA), and extends both methods to apply to the ICU network model. Such extensions are necessary due to special properties of the ICU network model that do not exist in other types of systems.

To illustrate the usefulness of the proposed QoS approximation methods, these methods are applied to the following optimization problem: given a network of ICUs, each with a predetermined capacity, find the optimal reservation thresholds for each ICU so that the overall blocking probability of external emergency patients is minimized, subject to maintaining a minimum QoS for internal emergency and elective patients. The following demonstrates that the accuracy and fast running time of the approximations of the present embodiment, as compared to simulation, allows for efficient coverage of large search spaces. Numerical results show that the proposed threshold reservation policy in one embodiment of the invention, with the reservation thresholds optimized using the proposed QoS evaluation method in the embodiment of the invention, produces much lower blocking for external emergency patients than the virtual ICU policy of Litvak (with the number of virtual ICU beds optimized using their QoS evaluation method).

### 2 Queueing Theory and Healthcare

Queueing theory has been used in the field of healthcare not only to analyze system performance, but also in order to facilitate system design. This section reviews several concepts from queueing theory which motivate both the ICU network model which the present invention seeks to optimize and the patient referral policy for this model in the invention.

### 2.1 Modeling Patient Flows

Newell DJ (1954) Provision of emergency beds in hospitals. British Journal of Preventive and Social Medicine 8(2):77-80 studied arrivals of emergency cases to a teaching hospital in England over the years of 1950 to 1952, and found the daily tallies could be modeled using a Poisson distribution, as long as Sunday and weekday arrivals were counted separately. In practice, many analytical models of patient arrivals to hospitals ignore daily or seasonal variations in the arrival rate, thus assuming a simple Poisson process. The same assumption applies to the method of the present embodiment. Furthermore, Litvak found that the QoS of their ICU network did not significantly depend on the patient LoS distribution apart from its mean, allowing a simple exponential LoS distribution to be used.

By assuming Poisson arrivals and exponential LoS, the state of an ICU network can be modeled as a continuous-time Markov chain, from which QoS measures can be (in theory) obtained via an exact analytical solution. On the other hand, since the number of states grows exponentially as the number of ICUs increases, exact analysis of the resulting state space is not a scalable analytical approach.

### 2.2 Resource Allocation and Threshold Reservation in a Network of Physically Separate ICUs

The present embodiment of the invention provides the use of a threshold reservation scheme. The threshold policy is such that external emergency patients cannot use the last few remaining beds of each ICU. Such a policy bares similarities to trunk reservation in telecommunications networks, in which the last few remaining circuits of each trunk are reserved for direct traffic in order to prevent overflow traffic (which use longer and more resource-intensive alternate routes) from dominating the network. Note that in trunk reservation, no individual circuit is explicitly reserved for direct traffic; likewise, in the proposed embodiment of the threshold reservation policy, no individual bed is explicitly reserved for a particular patient type or set of patient types. Threshold reservation thus maximizes resource sharing in periods of non-congestion, while protecting the QoS of internal emergency and elective patients, which cannot overflow, during periods of congestion.

### 2.3 QoS Approximation in Overflow Loss Systems

The ICU network model considered in some embodiments of the present invention belongs to a broad class of stochastic models known as overflow loss systems. In an overflow loss system, there is a set of request types and a set of server groups, each server group serves some subset of the request types in the system, and a routing policy determines the order which requests of each type attempt the set of accessible server groups for that request type. In the following description, only routing policies where these orderings are fixed (as opposed to state-dependent or random) is considered.

The classical analytical approximation approach for performance evaluation in overflow loss systems, known by various names such as the reduced load approximation, Erlang fixed-point approximation, and exponential decomposition, is known in teletraffic theory. In the following, the term exponential decomposition (ED) is used. ED decomposes the system into independent Erlang B subsystems by adding two simplifying assumptions to the analytical model: (i) that the offered traffic to each subsystem, composed of both direct and overflow traffic, is Poisson, and (ii) that the offered traffic to each subsystem is independent of all other traffic streams. Due to these two simplifying assumptions, ED dramatically reduces the computing time compared to exact analysis of the full state space. However, as these two assumptions may not always be valid, they can also lead to large approximation errors in various scenarios.

Moment matching has been proposed for reducing errors caused by the Poisson assumption. This approach was used effectively by Litvak for performance evaluation of their ICU network model under the virtual ICU policy. In the present embodiment, the method uses moment matching to provide conservative QoS estimates for the two patient types without overflow, namely internal emergency and elective patients.

On the other hand, moment matching provides only marginal improvement over traditional ED in systems involving mutual overflow, where the independence assumption forms the main source of error. Therefore, ED with moment matching is not adequate for QoS evaluation of external emergency patients under out proposed patient referral policy. Other ways to reduce errors caused by the independence assumption includes applying the technique used in traditional ED, i.e. decomposing the systems into independent Erlang B subsystems, on a surrogate of the original system. Ideally, the QoS of the surrogate closely approximates that of the original system, but the surrogate possesses certain properties which greatly reduce its approximation error caused by decomposition. The estimated QoS of the surrogate is then used as a QoS estimate for the original system.

The present embodiment adapts and extends one such surrogate-based approximation framework, the Information Exchange Surrogate Approximation (IESA) framework, to the proposed ICU network model, where it is used to evaluate the QoS of external emergency patients. IESA features an information exchange mechanism in which incoming calls/requests may exchange certain congestion information with calls/requests in service. This mechanism can capture traffic dependence in the system, and hence it can provide significantly improve the accuracy over ED. Numerical results demonstrate that IESA is more accurate and robust than traditional ED for the QoS evaluation of external emergency patients, while being much more computationally efficient than exact analysis or simulation. In fact, IESA has a closed form solution, whereas ED does not, due to the hierarchical nature of the information exchange mechanism.

### 3 Model

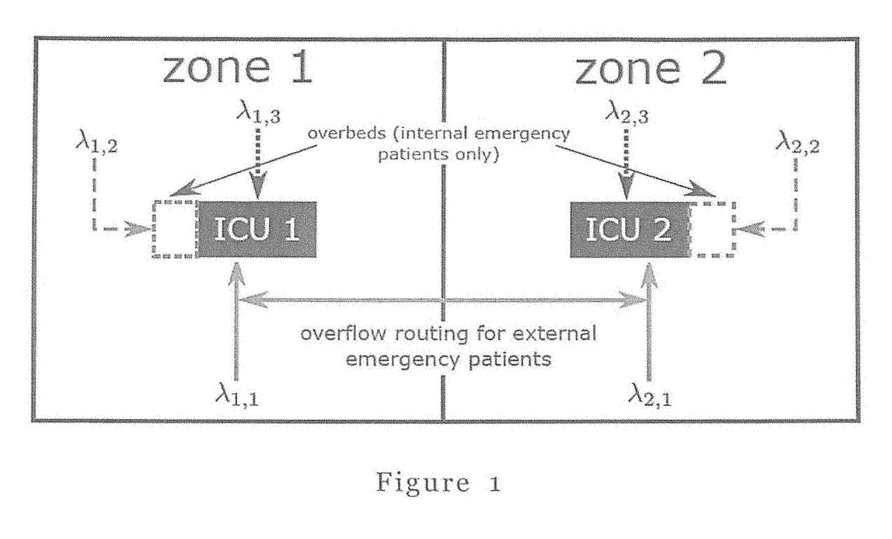

Consider the three-patient-type model of Litvak with external emergency patients, internal emergency patients, and elective patients, as depicted in FIG. 1. In this model, there are G ICUs, each with its own catchment zone. Let Zone i denote the catchment zone/area for ICU i. External emergency patients to each ICU i arrive from Zone i according to a Poisson process with rate λ_{i,1} and may be admitted to any ICU in the network. Such patients are blocked (i.e., transferred to another hospital network or demoted to a lower level of care) if and only if every bed in the entire ICU network is either occupied or reserved for other patient types.

Internal emergency patients and elective patients arrive directly at each ICU i in accordance to Poisson processes with rates λ_{i,2} and λ_{i,3}, respectively, and are not allowed to overflow. An internal emergency patient which cannot be admitted to a regular ICU bed will trigger the creation of a temporary overbed; in a physical ICU network, this may be a bed in another hospital department such as a post-anesthesia care ward or a separate, specialized cardiac ICU. Elective patients, on the other hand, generally correspond to non-time critical surgical operations; if no ICU is available for such patients, the operation is deferred. For simplicity, retrials will not be modeled; instead, any subsequent attempt of an elective patient to obtain an ICU bed is modeled as a new arrival.

Let Cᵢ denote the number of regular beds in ICU i, i.e. the rated capacity of that ICU. As in Litvak, in the present embodiment, assume that patient LoS is exponentially distributed with equal mean (except in Section 4, where it is shown, via simulation, that the QoS is not very sensitive to the shape of the LoS distribution apart from its mean). Without loss of generality, the mean is assumed here to be one.

As an analytical model, the Litvak model contains several simplifications compared to a physical ICU network, as listed in Table 1. Nevertheless, the model forms a good environment for testing new concepts and methodologies before they are applied to more complex real-world systems.

TABLE 1
Comparison of the ICU network model to a physical system.

| Physical network | Analytical model | Justification |
|---|---|---|
| Patients arrive to the ICU | The ICU network is treated | Poisson processes are well |
| network from other | as an isolated system to | suited to modeling events |
| hospital departments, | which patients arrive | that are rare from an |
| including the AED and | directly, according to a | individual point of view, |
| surgical units. The arrival | Poisson processes with | but which occur within a |
| process to the ICU | constant rate. | large population. Isolating |
| network is unknown. | | the ICUs from the rest of |
| | | the hospital network |
| | | simplifies analysis. |
| Patient LoS has an | Patient LoS is modeled | The sensitivity of the QoS |
| unknown distribution. | using an exponential | to the LoS distribution is |
| | distribution. | demonstrated in Litvak |
| | | and Section 4 to be low. |
| Certain ICUs may be | All ICU beds are | Simplification of the |
| better-equipped to deal | considered identical. Any | analytical model. |
| with certain patients, | penalty incurred by | |
| based on the types of | serving an external | |
| specialists required. | emergency patient at a | |
| | nonpreferred ICU (in the | |
| | form of transportation | |
| | costs, decreased quality of | |
| | care, etc.) is ignored. | |
| Elective patients are | Subsequent service | Simplification of the |
| deferred if no ICU bed is | attempts by elective | analytical model. |
| available and will | patients are treated as new | |
| reattempt their planned | arrivals. | |
| operation a later time. | | |
| Internal emergency | Internal emergency | The patient may require |
| patients may be referred | patients arriving at an ICU | increased resources |
| to another hospital unit, | create temporary overbeds | compared to a regular |
| e.g. the post-anesthesia | within the ICU itself when | patient despite being |
| care unit, if the ICU is full. | the ICU is full. | referred to a non-ICU unit. |
| | | Additionally, it is expected that |
| | | such patients will be |
| | | transferred back to the |
| | | ICU as soon as an ICU bed |
| | | becomes available. |

### 3.1 Notation for QoS Evaluation

For measuring the QoS of an ICU network, let Bᵢ denote the blocking probability of external emergency patients from catchment zone i, defined as the probability that such a patient is refused by all the ICUs in the network and thus rejected from the ICU network entirely. Let Dᵢ denote the deferral probability of elective patients arriving at ICU i, defined as the probability that the planned operation of an elective patient is deferred due to a lack of beds at ICU i. Let Tᵢ denote the mean number of overbeds at ICU i for internal emergency patients. Let B, D, and T represent the overall blocking probability, deferral probability, and mean number of overbeds for the entire network; thus

$$B = \frac{{\sum }_{z = 1}^{G} {λ}_{z , 1} {B}_{i}}{{\sum }_{z = 1}^{G} {λ}_{z , 1}}$$

$$D = \frac{{\sum }_{i = 1}^{G} {λ}_{i , 3} {D}_{i}}{{\sum }_{i = 1}^{G} {λ}_{i , 3}}$$

$$and$$

$$T = {\sum }_{i = 1}^{G} {T}_{i}$$

Finally, let bᵢ denote the congestion probability of ICU i for external emergency patients, defined as the probability that an external emergency patient arriving at ICU i will be refused by that ICU. The notation defined above is summarized in Table 2.

TABLE 2
Table of notations for the ICU network model

| Symbol | Definition |
|---|---|
| G | Number of ICUs in the system |
| λ_{i,1} | Arrival rate of external emergency patients from catchment |
| | zone i |
| λ_{i,2} | Arrival rate of internal emergency patients to ICU i |
| λ_{i,3} | Arrival rate of elective patients to ICU i |
| Cᵢ | Rated capacity of ICU i |
| B | Overall blocking probability of external emergency patients |
| | in the ICU network |
| Bᵢ | Blocking probability of external emergency patients from |
| | catchment zone i |
| bᵢ | Probability that an external emergency patient attempting ICU i |
| | will be refused by that ICU |
| T | Mean number of temporary overbeds in the ICU network for |
| | internal emergency patients |
| Tᵢ | Mean number of temporary overbeds for internal emergency |
| | patients in ICU i |
| D | Overall deferral probability of elective patients in the ICU |
| | network |
| Dᵢ | Deferral probability of elective patients at ICU i |

### 3.2 Virtual ICU Policy

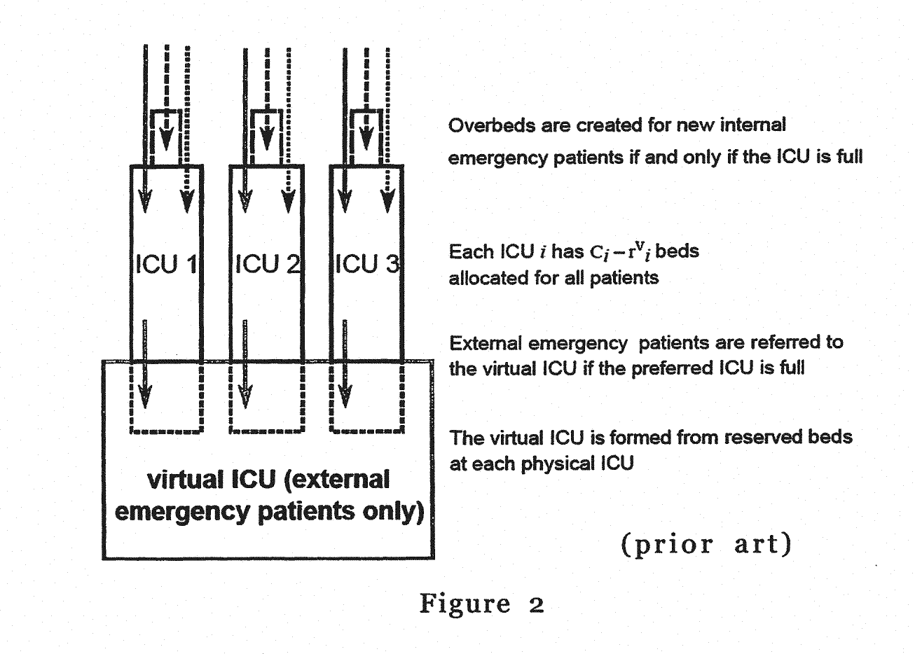

Section 3.4 compares the ICU policy in the present embodiment with the virtual ICU policy of Litvak. The virtual ICU policy was introduced by Litvak as a more efficient policy than a set of G fully independent ICUs, demonstrating that through resource sharing, improvements in QoS could be obtained for all patient types. Under the virtual ICU policy, each ICU i, i=1, . . . , G, reserves rᵢ^{V} beds exclusively for external emergency patients. These reserved beds form a virtual ICU which only serves external emergency patients. An external emergency patient m arriving from Zone i will first attempt to obtain one of the Cᵢ−rᵢ^{V} unreserved beds at ICU i. If none of these beds are available, the patient will attempt to obtain a bed at the virtual ICU. If all virtual ICU beds are also occupied, then the patient is blocked. A graphical depiction of the virtual ICU model is shown in FIG. 2.

Litvak provided a moment-matched version of ED for QoS evaluation under the virtual ICU policy, and demonstrated that this method produces accurate QoS results for this policy. In general, moment-matched ED is effective for hierarchical overflow models. On the other hand, the virtual ICU policy is sub-optimal in terms of maximizing QoS: the purely hierarchical structure of the virtual ICU model means that the level of resource sharing remains far from ideal.

### 3.3 Threshold Reservation Policy

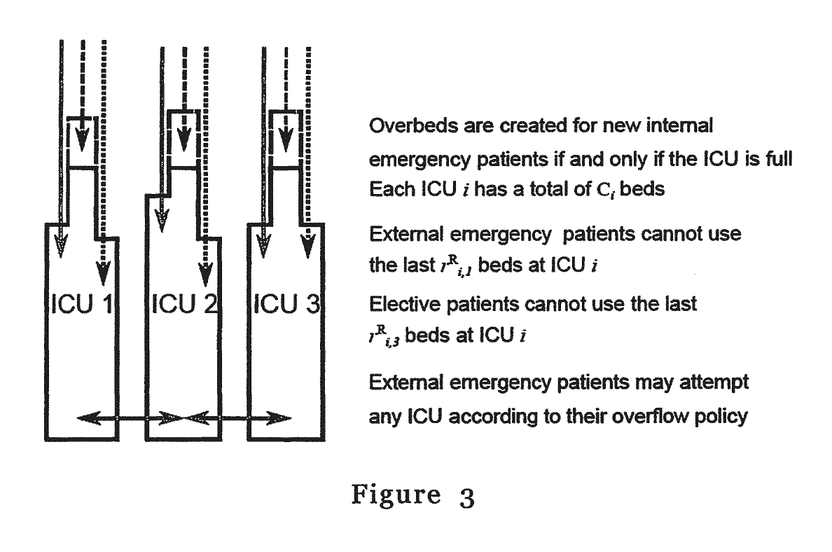

Let Γ_{z,n} denote the ICU to which external emergency patients from Zone z and with n previous service attempts are referred. Under the threshold policy, external emergency patients arriving from Zone z will attempt each bed in Γ_{z}=(Γ_{z,0}, Γ_{z,1}, . . . Γ_{z,G-1}) in order until an available ICU bed is found. Define z to be the overflow policy of external emergency patients from zone z. However, unlike in the virtual ICU model in Litvak, no beds are explicitly set aside for any patient type. Instead, the present embodiment impose a set of thresholds, r_{i,1}^{R} and r_{i,3}^{R} so that for each ICU i, i=1, . . . , G, external emergency patients are barred from last r_{i,1}^{R} beds and elective patients barred from last r_{i,1}^{R} from last r_{i,3}^{R} beds (i.e. these patients will not be admitted if the number of vacant beds at ICU i falls below the specified threshold). A graphical depiction of the threshold policy in one embodiment of the invention is shown in FIG. 3. Finally, let Γ=(Γ₁, Γ₂, . . . , Γ_{G}) denote the overflow policy of the entire network.

### 3.4 Numerical Comparison of Reservation Policies

In the following, consider an ICU network with 3 ICUs, with 20 beds in each ICU. The offered load for external emergency patients from Zone i is λ_{i,1}=λ and the offered load for internal emergency and elective patients to ICU i is λ_{i,2}=λ_{i,3}=λ. The overflow policy for external emergency patients is Γ=((i, 2, 3), (2, 3, i), (3, 1, 2)). The network is thus symmetrical in both offered load and overflow policy. In the following, the reservation policy is restricted to be the same for each ICU.

For each reservation setting, the QoS of the ICU network is evaluated using Markov-chain simulation. Simulation is terminated when either the 95% confidence interval, as computed using Student's t-distribution, lies within 1% of the simulation mean, or when thirty simulation runs have been completed. The few cases where the confidence interval does not fall within 1% of the simulation mean, even after thirty runs, all have the property of B<10⁻⁴. Such cases do not affect the results of this subsection, as the simulation error is dominated by the difference in QoS between the different reservation settings.

### 3.4.1 Minimizing the Blocking Probability of External Emergency Patients

For each λ in {5, 5.2, . . . , 6}, determine via simulation the optimal reservation settings to minimize the blocking probability B of external emergency patients, subject to T<0.3 and D<0.25. The results, shown in Table 3, demonstrate that the threshold policy reduces the blocking probability of external emergency patients from 60 to 85% compared to the virtual ICU policy. In addition, when restricting r_{i,1}^{R}=r_{i,3}^{R} for better comparison with the virtual ICU policy (which has only one reservation setting for each ICU), the threshold policy still results in lower blocking probability than the virtual ICU policy for λ>5.2, with the benefits of the threshold policy increasing with A. This demonstrates the increased level of resource sharing in the threshold policy, compared to the virtual ICU policy, has a large effect on the QoS of the network.

TABLE 3
Optimal reservation settings for minimizing the blocking probabilities of
external emergency patients in a symmetric 3-ICU network. Each entry
shows the blocking probability B of external emergency patients and
the corresponding reservation settings.

| | | 1 threshold | |
|---|---|---|---|
| | 2 thresholds | Threshold | Virtual |
| λ | Threshold policy | policy | ICU policy |
| 5 | 7.07 × 10⁻⁵ | 0.00138 | 0.00048 |
| | r_{i,1}^{R} = 0, r_{i,3}^{R} = 3 | no reservation | rᵢ^{V} = 4 |
| 5.2 | 0.00015 | 0.00259 | 0.00101 |
| | r_{i,1}^{R} = 0, r_{i,3}^{R} = 3 | no reservation | rᵢ^{V} = 4 |
| 5.4 | 0.00067 | 0.00453 | 0.00558 |
| | r_{i,1}^{R} = 0, r_{i,3}^{R} = 2 | no reservation | rᵢ^{V} = 3 |
| 5.6 | 0.00281 | 0.00752 | 0.00934 |
| | r_{i,1}^{R} = 0, r_{i,3}^{R} = 1 | no reservation | rᵢ^{V} = 3 |
| 5.8 | 0.00455 | 0.0172 | 0.0323 |
| | r_{i,1}^{R} = 0, r_{i,3}^{R} = 1 | no reservation | rᵢ^{V} = 2 |
| 6 | 0.0174 | 0.0174 | 0.0441 |
| | no reservation | no reservation | rᵢ^{V} = 2 |

Note that the optimal blocking probability for the two-threshold policy and virtual ICU policy is not continuous in λ, as would be the case if all solutions for a particular policy used the same reservation settings. As λ increases, certain reservation settings that are viable for lower λ become no longer viable as the constraints on T and D are no longer met. Note also that the optimal blocking probability for all policies is quite sensitive to the value of λ.

### 3.4.2 Minimizing the Overall Rejection Rate

For each λ in {5, 5.2, . . . , 6.0}, determine, via simulation, the optimal reservation settings to minimize the overall rejection rate, subject to B<0.05, T<0.3 and D<0.25. The overall rejection rate is defined as the proportion of patients that are either blocked or deferred:

$$\frac{mean rejection rate}{mean offered load} = \frac{B λ + D λ}{3 λ} = \frac{B + D}{3} .$$

The results, shown in Table 4, demonstrate a 32%-44% decrease in rejection rate by adopting the threshold policy instead of the virtual ICU policy. In addition, comparison of the QoS demonstrates that the threshold policy can result in improved service for all three patient types compared to the virtual ICU policy. Finally, when restricting r_{i,1}^{R}=r_{i,3}^{R} for better comparison with the virtual ICU policy (which has only one reservation setting for each ICU), the threshold policy still gives a lower overall rejection rate than the virtual ICU policy.

TABLE 4
Optimal reservation settings for minimizing the overall rejection rate of a
symmetric 3-ICU network. Each entry shows the overall rejection rate of
the ICU network, the QoS of each patient type, and the corresponding
reservation settings.

| λ | Threshold policy | Virtual ICU policy |
|---|---|---|
| 5 | 0.02391 | 0.04314 |
| | B = 0.00133 | B = 0.00552 |
| | T = 0.06127 | T = 0.1158 |
| | D = 0.06774 | D = 0.1129 |
| | no reservation | rᵢ^{V} = 2 |
| 5.2 | 0.03144 | 0.05391 |
| | B = 0.00255 | B = 0.00937 |
| | T = 0.08248 | T = 0.1441 |
| | D = 0.08669 | D = 0.1336 |
| | no reservation | rᵢ^{V} = 2 |
| 5.4 | 0.04069 | 0.06670 |
| | B = 0.00453 | B = 0.0149 |
| | T = 0.1083 | T = 0.1762 |
| | D = 0.1085 | D = 0.1554 |
| | no reservation | rᵢ^{V} = 2 |
| 5.6 | 0.05174 | 0.08177 |
| | B = 0.00752 | B = 0.0225 |
| | T = 0.1300 | T = 0.2116 |
| | D = 0.1327 | D = 0.1779 |
| | no reservation | rᵢ^{V} = 2 |
| 5.8 | 0.06471 | 0.09911 |
| | B = 0.001172 | B = 0.03215 |
| | T = 0.1741 | T = 0.2504 |
| | D = 0.1590 | D = 0.2009 |
| | no reservation | rᵢ^{V} = 2 |
| 6 | 0.07977 | 0.11885 |
| | B = 0.01472 | B = 0.04409 |
| | T = 0.2143 | T = 0.2927 |
| | D = 0.1870 | D = 0.2243 |
| | no reservation | rᵢ^{V} = 2 |

### 3.4.3 Example where Restricting Overflow Lowers the Overall Rejection Rate

Litvak found that internal emergency patients have a lower arrival rate than the other patient types. Thus, consider the same optimization problem as in Section 3.4.1 but with λ_{i,1}=λ and λ_{i,2}=λ−1. The results, shown in Table 5, demonstrate a 20%-43% decrease in rejection rate by adopting the threshold policy instead of the virtual ICU policy. In Table 5, the superscript R in r_{i,1}^{R} refers to the policy in the present embodiment; superscript V in r_{i,1}^{V} refers to the policy in Litvak. The results in Table 5 also demonstrate that setting r_{i,1}^{R}=r_{i,3}^{R}=0, i.e., no reservation, does not necessarily result in the lowest rejection rates, unlike in Section 3.4.2. This is despite maximal resource sharing in the sense that external patients have access to all beds in all ICUs in the network and no patient is ever barred from an ICU if there is at least one bed available. Instead, for λ=5 or 5.2, the method of the present embodiment obtains the counter-intuitive result of reduced overall rejection rate when the overflow of external emergency patients is restricted. This is because overflowing external emergency patients adversely affect the QoS of internal emergency and elective patients.

TABLE 5
Optimal reservation settings for minimizing the overall rejection rate of
a 3-ICU network, with reduced arrival rates of internal emergency
patients. Each entry shows the overall rejection rate of the ICU
network, the QoS of each patient type, and the corresponding
reservation settings.

| λ | Threshold policy | Virtual ICU policy |
|---|---|---|
| 5 | 0.01200 | 0.02119 |
| | B = 0.00246 | B = 0.00902 |
| | T = 0.01971 | T = 0.03973 |
| | D = 0.02862 | D = 0.05454 |
| | r_{i,1}^{R} = 1, r_{i,3}^{R} = 0 | rᵢ^{V} = 1 |
| 5.2 | 0.01789 | 0.02775 |
| | B = 0.00492 | B = 0.01419 |
| | T = 0.02842 | T = 0.05340 |
| | D = 0.03893 | D = 0.06905 |
| | r_{i,1}^{R} = 1, r_{i,3}^{R} = 0 | rᵢ^{V} = 1 |
| 5.4 | 0.02542 | 0.03550 |
| | B = 0.00160 | B = 0.02128 |
| | T = 0.05521 | T = 0.06987 |
| | D = 0.07144 | D = 0.08523 |
| | no reservation | rᵢ^{V} = 1 |
| 5.6 | 0.03325 | 0.04439 |
| | B = 0.00298 | B = 0.03039 |
| | T = 0.07406 | T = 0.08907 |
| | D = 0.09079 | D = 0.1028 |
| | no reservation | rᵢ^{V} = 1 |
| 5.8 | 0.04275 | 0.05441 |
| | B = 0.00517 | B = 0.04162 |
| | T = 0.09702 | T = 0.1111 |
| | D = 0.1127 | D = 0.1216 |
| | no reservation | rᵢ^{V} = 1 |
| 6 | 0.05406 | 0.06807 |
| | B = 0.00839 | B = 0.02555 |
| | T = 0.1241 | T = 0.1831 |
| | D = 0.1370 | D = 0.1786 |
| | no reservation | rᵢ^{V} = 2 |

### 3.4.4 Robustness to Increases in the Offered Load

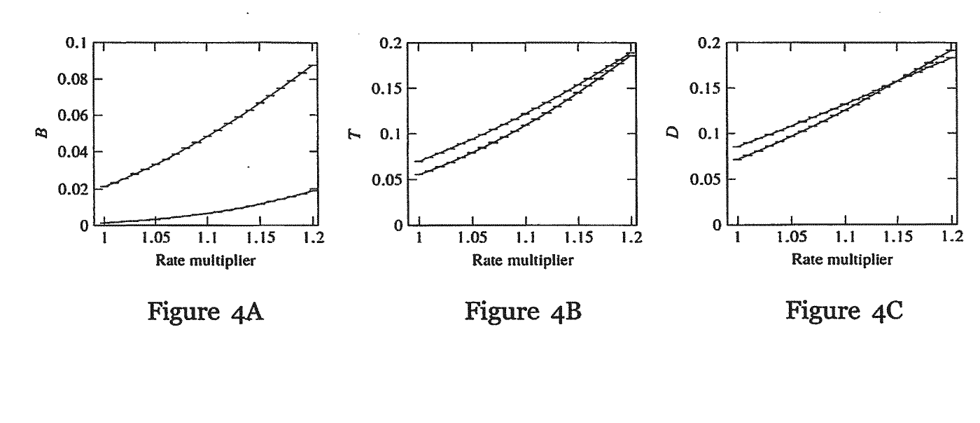

Certain events such as the outbreak of an infectious disease may cause short-term spikes in the arrival rate of patients to an ICU network. In order to demonstrate that the benefits of the threshold policy over the virtual ICU policy are not dependent on the offered load, in one example, consider the λ=5.4 case from Table 3. Using the optimal reservation settings for both policies for λ=5.4, examine the effect on B, T, and D as the offered load is increased by up to 20%. The results are shown in FIGS. 4A-4C. As the offered load increases, the gap in B between the threshold policy and the virtual ICU policy also increases. On the other hand, T and D are about the same for both policies. In other words, the threshold policy is robust to increases in the offered load in the sense that the threshold policy continues to achieve a better QoS than the virtual ICU policy when the arrival rates are increased (with the reservation settings fixed).

### 4 Sensitivity to the Patient Length-of-Stay Distribution

Litvak demonstrated via simulation that their ICU network model, using their virtual ICU policy, is not very sensitive to the shape of the patient LoS distribution apart from its mean. This section shows that this near insensitivity also applies to the threshold policy. Consider the same 3-ICU network as Section 3.4 and generate 1000 random configurations, with 5.0≤λ_{i,t}≤6.0, 0≤r_{i,1}^{R}≤3 and 0≤r_{i,3}^{R}≤3 for each i=1, 2, . . . G and t=0, 1, 2. The number of simulation runs is such that the 95% confidence interval, as computed using Student's t-distribution, lies within 1% of the simulation mean.

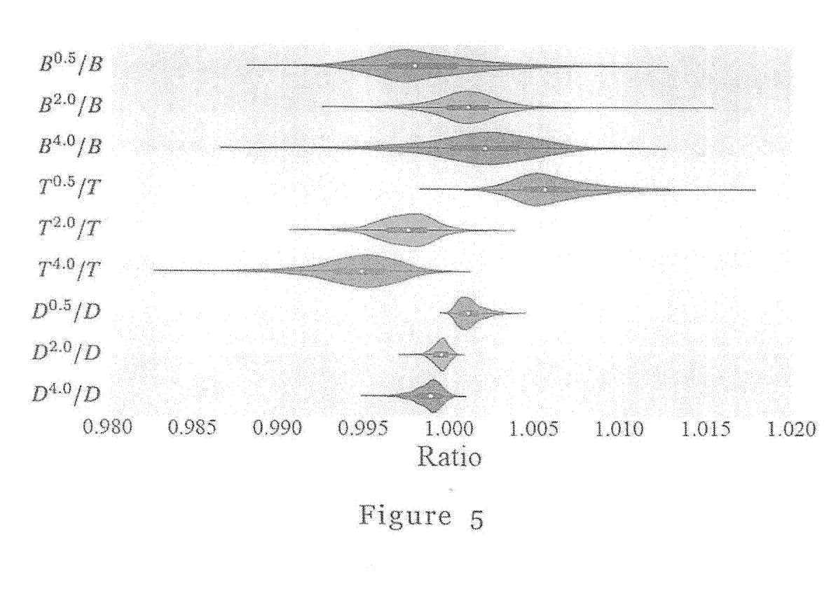

Let B^{x}, T^{x}, and D^{x} denote the blocking probability of external emergency patients, mean number of overbeds for internal emergency patients, and deferral probability of elective patients, respectively, for a lognormal LoS distribution with mean 1.0 and variance x, as found by simulation; and B, T, and D the same for an exponential LoS distribution, also with a mean of 1.0. The distributions of the ratios B^{x}/B, T^{x}/T and D^{x}/D are shown in FIG. 5 for xϵ{0.5, 2.0, 4.0}.

The results suggest that the QoS of the ICU network used in the present example is not very sensitive to the patient LoS distribution, with all results within the interval [0.98, 1.02].

### 5 Estimating the QoS of an ICU Network

While accurate approximations exist for ICU networks using the virtual ICU policy in Litvak, the presence of mutual overflow under the threshold policy means that estimation of QoS becomes considerably more difficult. Additionally, although there are similarities between the ICU network and other overflow systems such as telecommunications systems and call centers, there are also some fundamental differences. For example, the ICU network of the present example considers three different patient types, of which only one type may overflow. Furthermore, the concept of an overbed is unique to the current ICU network model. These differences make the problem faced by the inventors of the present invention challenging. In this section, several approximations for QoS in an ICU network under the threshold policy is examined and compared. It is also shown that how they can be extended to apply to the current ICU network model of the present example.

### 5.1 Markov Chain Representation of a Single ICU

In this example, first start by the simplifying assumption that all traffic offered to an ICU, including overflow traffic, follows a Poisson process. Let a_{i,n} denote the offered traffic of external emergency patients from Zone i which have overflowed n times in the network. Then

a_{z,0}=λ_{z,1}

a_{z,n}=a_{z,n-1}b_{Γ}_{_{z,n-1}},n>0 (1)

Let xᵢ denote the total offered traffic of external emergency patients to ICU i. Then

$${x}_{i} = {\sum }_{z = 1}^{G} {\sum }_{n : {Γ}_{z , n} = i}^{ } {a}_{z , n} . \qquad ( 2 )$$

Thus, ICU i receives a total offered load of xᵢ+λ_{i,2}+λ_{i,3} Erlangs.

By assuming that the arrival process to each ICU is a Poisson process, obtain a one-dimensional Markov chain representation for each ICU i, i=1, . . . , G, as follows. Let state j denote the state in which there are j patients in service, and q_{j,k} be the transition rate from state j to state k. Then

q_{j,j+1}=xᵢ1{j<Cᵢ−r_{i,1}^{R}}+λ_{i,2}+λ_{i,3}1{j<Cᵢ−r_{i,3}^{R}}

q_{j,j-1}=j

q_{j,k}=0,|j−k|≠1

where 1{⋅} represents the indicator function.

From the transition rate matrix q_{[j,k]}, the probability of each state j, jϵN, denoted as πⱼ, can be obtained. Then

$${b}_{i} = {\sum }_{j = {C}_{i} - {r}_{i , 1}^{R}}^{ } {π}_{j} \\ {T}_{i} = {\sum }_{j = {C}_{i} + 1}^{∞} {π}_{j} ( j - {C}_{i} ) \\ {D}_{i} = {\sum }_{j = {C}_{i} - {r}_{i , 3}^{R}}^{ } {π}_{j} \qquad ( 3 )$$

### 5.2 Exponential Decomposition (ED)

Exponential Decomposition (ED) can be applied to the ICU network by treating xᵢ for each ICU i, i=1, 2, . . . , G, as mutually independent. This results in a system of fixed-point equations involving (xᵢ)ᵢ₌₁^{G} and (bᵢ)ᵢ₌₁^{G}, which can be solved via iterative substitution using equations (1)-(3). The stopping criterion is defined as follows. Let bᵢ^{(k)} denote the k^{th}-iteration estimate of bᵢ. The fixed-point iteration is terminated when |bᵢ^{(k)}−bᵢ^{(k-1)}<10⁻⁸ for all i=1, 2, . . . , G.

After obtaining xᵢ and bᵢ for each ICU i, i=1, 2, . . . , G, the quantity Bᵢ can be obtained as the product of the congestion probabilities for each ICU in Γᵢ, i.e., Bᵢ=Π_{jϵΓ}_{ᵢ}bⱼ.

### 5.3 Information Exchange Surrogate Approximation (IESA)

Information Exchange Surrogate Approximation (IESA) is based on the applying the underlying methodology of ED, namely decomposition of the ICU network into a set of independent queues with Poisson input, to a surrogate model of the original network, so that the dependencies between ICUs are represented in a manner that is preserved when decomposition is applied. In the IESA surrogate model, each external emergency patient carries three attributes: z, the originating zone, Δ, the set of attempted ICUs, and Ω, an estimate of the number of ICUs in the network currently refusing external emergency patients. All new patients start with Δ=ø; and Ω=0. Use the term (z, Δ, Ω)-patient to denote an external emergency patient from Zone z which has attempted each ICU in Δ and has a congestion estimate of Ω. Unlike the “true” model of the ICU network, in addition to blocking if all ICUs have been attempted unsuccessfully, external emergency patients in the IESA model will also abandon the network if Ω reaches G.

Consider a (z₁, Δ₁, Ω₁)-patient attempting ICU i. If a bed is available at ICU i for external emergency patients, the patient is admitted. Otherwise, the patient is compared to the external emergency patient with the highest Ω values among all external emergency patients residing at ICU i, which is denoted as an (z₂, Δ₂, Ω₂)-patient. Ties are broken arbitrarily. If Ω₁≥Ω₂, then the incoming patient overflows normally and becomes a (z₁, Δ₁∪{i}, Ω₁+1)-patient. On the other hand, if Ω₁<Ω₂, then exchange of Ω occurs and the incoming patient overflows as an (z₁, Ω₁∪{i}, Ω₂+1)-patient, while the admitted patient becomes an (z₂, Δ₂, Ω₁)-patient. Note that due to these rules, Ω≥|Δ| for all incoming patients.

IESA thus forms a hierarchical traffic structure based on Ω, a where level j of the hierarchy includes all patients with Ω less than or equal to j. Due to abandonment when Ω=G, the hierarchy has a total of exactly G layers, from 0 to G−1. Due to this hierarchy, IESA does not require fixed-point iterations when applied to the ICU network model of the present example, unlike EFPA.

Let:
e_{z,n,j} denote the offered traffic to ICU Γ_{z,n} composed of external emergency patients from Zone z which have overflowed n times in the network and have a congestion estimate of j;
\tilde{e}_{z,n,j} denote the offered traffic to ICU Γ_{z,n} composed of external emergency patients from Zone z which have overflowed n times in the network and have a congestion estimate of 0, 1, . . . or j;
a_{i,n,j} denote the offered traffic to ICU i composed of all external emergency patients which have overflowed n times in the network and have a congestion estimate of j;
ã_{i,n,j} denote the offered traffic to ICU i composed of all external emergency patients which have overflowed n times in the network and have a congestion estimate of 0, 1, . . . or j;
A_{i,j} denote the offered traffic to ICU i composed of all external emergency patients with congestion estimate 0, 1, . . . or j; and
b_{i,j} denote the congestion probability of ICU i for external emergency patients with congestion estimate 0, 1, . . . or j.

$${e}_{z , 0 , j} = \{ \begin{matrix}{λ}_{z , 1} & j = 0 \\ 0 , & otherwise ,\end{matrix} \\ {\tilde{e}}_{z , n , j} = {\sum }_{k = n}^{j} {e}_{z , n , k} , and \\ {\tilde{a}}_{i , n , j} = {\sum }_{k = n}^{j} {e}_{i , n , k} .$$

By definition,
Summing over all possible z,

$${a}_{i , n , j} = {\sum }_{z : {Γ}_{z , n} = i} {e}_{z , n , j} .$$

Summing over all possible n,

$${A}_{i , j} = {\sum }_{n = 0}^{G - 1} {\tilde{a}}_{i , n , j} .$$

From A_{i,j}, λ_{i,2}, and λ_{i,3}, b_{i,j} can be computed via Markov chain analysis as described in Section 5.1. In accordance with the information exchange mechanism,

e_{z,n,j}=e_{z,n-1,j-1}b_{Γ}_{_{z,n-1,j-1}}+e_{z,n-1,j-2}(b_{Γ}_{_{z,n-1,j-1}}−b_{Γ}_{_{z,n-1,j-2}}). (4)

The above values can be obtained iteratively for j=0, 1, . . . , G−1.

Finally, the blocking probability of external emergency patients in zone i is

$${B}_{i} = {\sum }_{n = 1}^{G - 1} {e}_{i , n , G} . \qquad ( 5 )$$

Note that equation (5) is a slight abuse of notation as patients with a congestion estimate of G are never offered to any ICU; however, defining e_{z,n,G} as per equation (4) yields the correct result for equation (5).

The values of Tᵢ and Dᵢ can be estimated from the last (i.e. G−1^{th}) level of the IESA hierarchy using the same Markov-chain analysis as for b_{i,G-1}.

### 5.4 Numerical Comparison of ED and IESA

Consider ICU networks of G=3, 4, or 5 ICUs. External emergency patients are referred to an ICU in a round-robin manner: thus an external emergency patient from zone i will attempt ICUs i, i+1 . . . , G, 1, 2, . . . i−1, in that order. For each value of G, 500 random configurations were generated with the following parameters:
15-20 beds in each ICU (i.e. 15≤Cᵢ≤20 for i=1, 2, . . . , G)
reservation thresholds r_{i,1}^{R} and r_{i,3}^{R} of 0 to 3 for each ICU i, and
arrival rates λ_{i,t} of 0.25Cᵢ to 0.3Cᵢ for each ICU i and for each patient type t.

The configurations were then filtered according to the following conditions:
a blocking probability B of between 0.1% and 5% for external emergency patients, as estimated by IESA;
at most 0.1G overbeds (i.e. T as estimated by IESA; and
a deferral probability D of at most 25% for elective patients, as estimated by IESA.

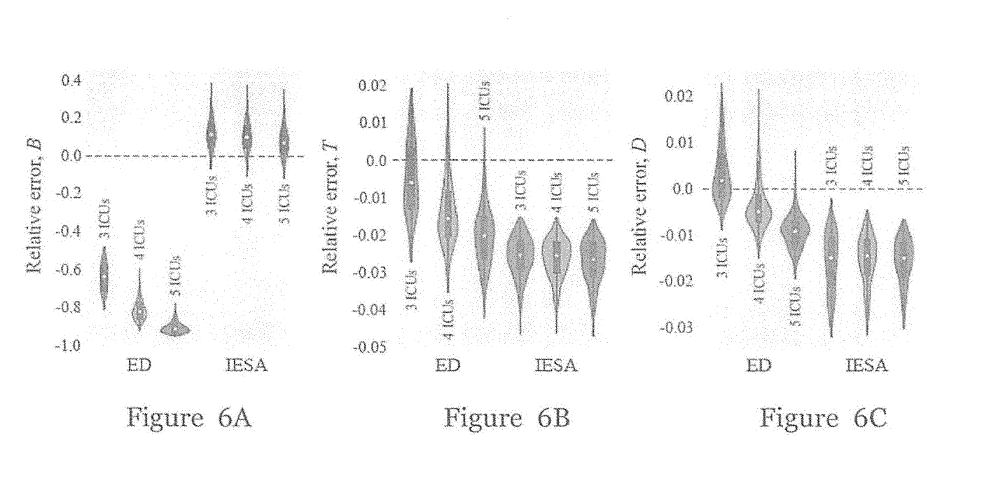

The number of valid configurations were 427, 452, and 372 for G=3, 4, and 5, respectively. For each valid configuration, B, T, and D were evaluated using Markov chain simulation, ED, and IESA. The number of simulation runs is such that the 95% confidence interval, as computed using Student's t-distribution, lies within 1% of the simulation mean. The relative errors of ED and IESA are shown in FIGS. 6A-6C. The results demonstrate that IESA is much more accurate than ED when estimating the blocking probability of external emergency patients. On the other hand, both approximations are fairly accurate for internal emergency and elective patients, with ED being slightly more accurate than IESA.

### 6 Obtaining a Conservative Estimate for Patient QoS

When dimensioning an ICU network, it is generally necessary to ensure that the QoS estimates for some patients are conservative. For example, if one of the optimization constraints is that the deferral probability D of elective patients must not exceed Dₘₐₓ, then any estimation of D must be equal to or greater than the actual value of D. This section presents a method of obtaining conservative estimates of T, the mean number of overbeds in the network, and D, the deferral probability of elective patients.

### 6.1 Hayward's Approximation

Overflow traffic in overflow loss systems generally has a higher peakedness (variance-to-mean ratio) than fresh traffic, and such peakedness increases the blocking probability of requests offered to the system. For a G/M/N/N queue offered traffic with mean in and variance v, with z=v/m, a simple but effective blocking probability approximation is provided as:

$$B ( m , v , N ) = B ( \frac{m}{z} , \frac{N}{z} ) .$$

This is equivalent to splitting the system into z independent

$$G \text{/} M \text{/} \frac{N}{z} \text{/} \frac{N}{z}$$

queues, thus raising the blocking probability of the system as servers in different queues now cannot coordinate to reduce congestion in the system. In many cases, N/z will not be an integer; Jagerman DL (1984) Methods in traffic calculations. AT&T Bell Laboratories Technical Journal 63(7):1283-1310 gives an analytic continuation of the Erlang B function for such cases.

To adapt Hayward's approximation to an ICU network model with threshold reservation, in the present embodiment, construct a Markov chain as follows.

Let xᵢ be the offered load of external emergency patients to ICU i and let vᵢ be the corresponding variance. Then the total offered traffic to ICU i has mean Mᵢ=xᵢ+λ_{i,2}+λ_{i,3} and variance Vᵢ=vᵢ+λ_{i,2}+λ_{i,3}. Define zᵢ=Vᵢ/Mᵢ.

Split the ICU into zᵢ independent parts so that the offered load to each part contains Cᵢ/zᵢ beds and the offered traffic to each part composed of external emergency, internal emergency, and elective patients is Poisson with means a_{i,1}=xᵢ/zᵢ, a_{i,2}=λ_{i,2}/zᵢ, and a_{i,3}=λ_{i,3}/zᵢ, respectively. The reservation thresholds for external emergency and elective r_{i,1}^{R}/zᵢ and r_{i,1}^{R}/zᵢ, respectively.

Non-integer ICU sizes and reservation thresholds are handled as follows. As in Section 5.1, let state j denote the state in which there are j patients in service, and q_{j,k} be the transition rate from state j to state k. Let c_{i,1}=(Cᵢ−r_{i,1}^{R})/zᵢ, c_{i,2}=Cᵢ/Zᵢ and c_{i,3}=(Cᵢ−r_{i,3}^{R})/zᵢ. Let n_{i,t} and f_{i,t} be the integer and fractional parts of c_{i,t}, respectively, for t=1, 2, or 3. Furthermore, define

$${u}_{i , j , t} = \{ \begin{matrix}{a}_{i , t} , & j < {n}_{i , t} \\ {a}_{i , t} {f}_{i , t} , & j = {n}_{i , t} \\ 0 , & j > {n}_{i , t} .\end{matrix} \\ Then \\ {q}_{j , j + 1} = {u}_{i , j , 1} + {u}_{i , j , 2} + {u}_{i , j , 3} \\ {q}_{j , j - 1} = j \\ {q}_{j , k} = 0 , \vert j - k \vert \neq 1 ,$$

from which the steady-state probability of each state j can be obtained.

$${b}_{i} = ( 1 - {f}_{1} ) {π}_{{n}_{i , 1}} + {\sum }_{j = {n}_{i , 1}}^{∞} {π}_{j}$$

$${T}_{i} = {\sum }_{j = ⌈ {n}_{i , 2} ⌉}^{∞} {π}_{j} ( j - {n}_{i , 2} )$$

$${D}_{i} = ( 1 - {f}_{3} ) {π}_{{n}_{i , 3}} + {\sum }_{j = {n}_{i , 3}}^{∞} {π}_{j}$$

Finally,

### 6.2 Overflow Variance of External Emergency Patients

Let a=x_{i,k} denote the offered traffic to ICU i composed of external emergency patients that have overflowed k times in the system, and let v denote the corresponding variance. Let z=v/a. To estimate the overflow traffic of patients corresponding to this input stream, in this embodiment, construct an M/M/n/n queue offered a′=a/z Erlangs of Poisson traffic so that E (a′, n)=bᵢ. A method of computing n is given by Jagerman.

The overflow mean and variance of the imaginary queue are a′ₒᵤₜ=a′bᵢ and

$${v}_{out}^{′} = {a}_{out}^{′} [ 1 - {a}_{out}^{′} + \frac{{a}^{′}}{n - {a}^{′} + {a}_{out}^{′} + 1} ] ,$$

respectively.

Finally, the overflow variance from ICU i composed of emergency patients that have overflowed k+1 times is estimated as v′ₒᵤₜz.

### 6.3 Numerical Results

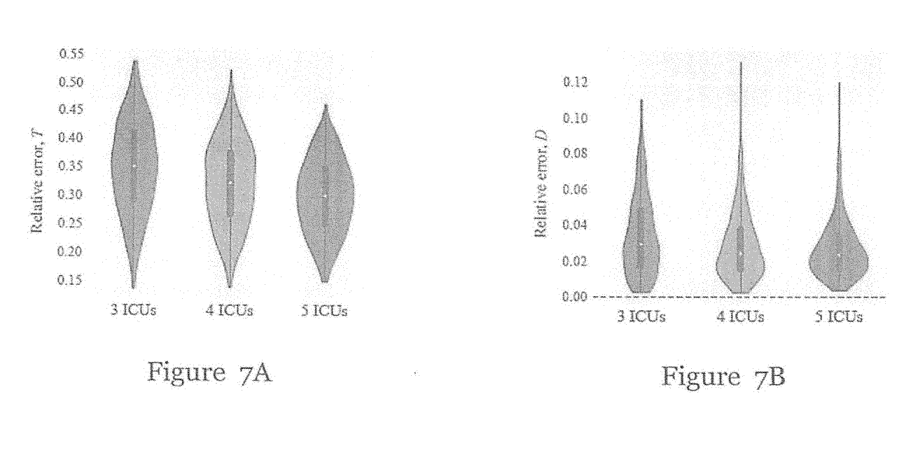

By using the methods described in Sections 6.1 and 6.2, in the present embodiment, it is possible to create a moment-matched version of ED, also called EDm. Using the same configurations as in Section 5.4, the results as shown in FIGS. 7A and 7B can be obtained, which demonstrate that EDm gives conservative estimates of T and D for the ICU network.

### 7 Minimizing the Blocking of External Emergency Patients

In one example, from the randomly generated configurations from Section 5.4, select 100 configurations for G=3 and G=4, and 48 configurations for G=₅, For each network, apply exhaustive search to solve the following problems:

$$\begin{matrix}{argmin}_{\{ {r}_{i , 1}^{V} , \vert i = 1 , … G ]} {B}_{V} \\ s . t . {T}_{V} < 0.1 G \\ {D}_{V} < 0.25 \\ ∀ i , {r}_{i , 1}^{V} \leq 10 \\ and & ( P1 ) \\ {argmin}_{\{ {r}_{i , 1}^{R} , {r}_{i , 3}^{R} \vert i = 1 , … G ]} {B}_{R} \\ s . t . {T}_{R} < 0.1 G \\ {D}_{R} < 0.25 \\ ∀ i , {r}_{i , 1}^{R} \leq 5 \\ ∀ i , {r}_{i , 3}^{R} \leq 5 , & ( P2 )\end{matrix}$$

where the subscripts V and R represent the virtual ICU policy and the proposed threshold reservation policy in the present embodiment, respectively.

Use IESA to approximate B_{R}, and EDm, which has been shown in Section 6.3 to be conservative, to approximate T_{R} and D_{R}. For the virtual ICU policy, use the approximation method defined in Litvak. Let B*_{V}, and B*_{R} denote the optimal values of B_{V} and B_{R}, respectively.

Table 6 shows both the mean and standard deviation of B*_{V}/B*_{R}, which represents the reduction in blocking probability of external emergency patients, using both approximation and Markov-chain simulation. The results demonstrate a much lower blocking probability of external emergency patients when the threshold reservation policy is used (i.e. B*_{R}<<B*_{V}), with the difference in performance between the two policies increasing with the number of ICUs in the network. Note that simulation in used here only to evaluate the QoS of the final configuration returned by the optimization process.

TABLE 6
Ratio of B_{V}* to B_{R}* for the optimal reservation settings for an ICU
network under the virtual ICU and threshold policies.

| | | |
|---|---|---|
| | B_{V}*/B_{R}* | B_{V}*/B_{R}* |
| Number | (estimated) | (simulated) |

| of ICUs | Mean | St. dev. | Mean | St. dev. |
|---|---|---|---|---|
| 3 | 3.806 | 0.7557 | 4.775 | 0.7487 |
| 4 | 8.716 | 1.601 | 11.79 | 1.691 |
| 5 | 19.34 | 4.381 | 28.24 | 4.463 |

Numerical results show that IESA is conservative for estimating B*_{R} for the optimal reservation setting in all cases considered. As ERM is very accurate at estimating B_{V} in ICU networks using the virtual ICU policy, the end result is that the estimation of B*_{V}/B*_{R} is also conservative for all cases considered. In addition, since EDm is conservative for estimating T_{R} and D_{R}, all solutions found for the threshold policy are valid. In other words, the approximate approach in the present embodiment provides not only a valid solution in all cases to optimization problem (P2), but also conservative estimates of the QoS and the amount of improvement achieved over the virtual ICU policy.

### 7.1 Running Times

For the optimization algorithm in the present embodiment for the threshold reservation policy, the running times for G=3, 4, and 5 are shown in Table 7. It is demonstrated that due to the speed of EDm and IESA, it is possible to perform exhaustive search for networks of up to 5 ICUs. The average speed as calculated for G=5 is 529.3 QoS evaluations per second (where one evaluation includes B, T, and D).

TABLE 7
Running times for optimizing reservation thresholds for networks of 3, 4,
and 5 ICUs.

Running time
(s)

| Number | | St. |
|---|---|---|
| of ICUs | Mean | dev. |
| 3 | 29.706 | 1.8479 |
| 4 | 1957.9 | 83.538 |
| 5 | 114243 | 3732.8 |

### 8 Conclusion

The above embodiments of the present invention have provided a threshold-based patient referral policy for the admission of patients to a network of ICUs. It has been shown that such new policy can achieve a higher patient acceptance level than the previously proposed policy in Litvak using a smaller number of beds, resulting in improved service for all patients. The proposed policy in the above embodiments incorporates important concepts and insights from traditional teletraffic theory, including the overflow loss model with multiple streams of calls (i.e. patients), resource sharing improved by allowing mutual overflow for overflow calls (i.e. external emergency patients), and trunk reservation for reserving the last unused amount of resource at each node (i.e. the last few unused beds at each ICU that external emergency patients cannot use) for providing sufficient service level for non-overflow calls (i.e. internal emergency and elective patients).

In particular, the above embodiments focus on the problem of minimizing the blocking probability of external emergency patients in an ICU network subject to meeting minimum QoS requirements for internal emergency and elective patients. This is achieved in three parts: (1) the proposed threshold-based policy for patient referral, (2) accurate and computationally efficient analytical approximation methods for estimating the QoS of an ICU network under the proposed policy, and (3) the incorporation of these approximation methods into an algorithm for quickly determining the optimal reservation thresholds for each ICU in the network. In addition to the combining existing concepts and methods to construct a comprehensive design, QoS evaluation, and optimization framework, new contributions include the construction of a new moment-matching method specific to the ICU network model with a threshold reservation policy and the unique combination of IESA and EDm to create a conservative analytical approximation method for all three patient types.

Numerical results demonstrate that the above embodiments of the threshold policy are more efficient than the virtual ICU policy of Litvak. This is because the proposed threshold policy can enhance the level of resource sharing by allowing an external emergency patient to be assigned to any ICU bed in the network as long as none of the vacancy thresholds are violated. On the other hand, under the policy of Litvak, an external emergency patient is only allowed to overflow to a dedicated group of ICU beds, thus limiting the level of resource sharing. Therefore, the proposed policy can achieve a lower blocking probability for external emergency patients than the previous policy given the same QoS requirements for internal emergency and elective patients. Alternatively, the threshold policy can reduce the overall rejection rate of an ICU network compared to virtual ICU policy (a reduction of 20-44% was achieved in the above numerical examples). Enhanced cooperation between hospitals by improving resource sharing can achieve a higher acceptance level with a smaller number of beds resulting in improved service for all patients in this scenario. On the other hand, numerical results demonstrate an unexpected phenomenon: maximizing resource sharing, by allowing external emergency patients to attempt any ICU and setting no reservation thresholds whatsoever, does not necessarily lead to the lowest overall patient rejection rate.

Numerical results also demonstrate that the QoS of ICU networks using the threshold policy is not very sensitive to the patient LoS distribution apart from its mean, meaning that the QoS approximations developed as part of the present invention are applicable to a wide range of QoS networks with different patient LoS distributions.

Numerical results also demonstrate that IESA can provide a much more accurate estimate of the blocking probability of external emergency patients than the classical method, ED. On the other hand, while ED by itself can achieve relatively accurate estimates of both the mean number of overbeds required for internal emergency patients and the mean deferral probability of elective patients, such estimates are not conservative, a requirement of the proposed optimization algorithm. The above description has therefore presented a version of ED incorporating moment-matching, namely EDm, which was demonstrated to be conservative in all the above numerical tests.

Optimization of ICU networks is performed in the above examples of the invention using a unique combination of IESA for the QoS evaluation of external emergency patients and EDm for the QoS evaluation of non-overflow patients (i.e. internal emergency and elective patients). The speed of IESA and EDm allows us to use exhaustive search for the proposed optimization algorithm for ICU networks of up to five ICUs. Numerical results demonstrate much better QoS (e.g. an average of 4.7, 11.7, and 28.2 times blocking probability reduction for external emergency patients in a 3-ICU, 4-ICU, and 5-ICU network, respectively) can be achieved by the proposed threshold policy, using the proposed approximation-based optimization algorithm, than for the virtual ICU policy, using the optimization method of Litvak. Additionally, the approximate approach in the above embodiments provides not only a valid solution in all cases for the threshold policy, but also conservative estimates of the QoS and amount of improvement achieved over the virtual ICU policy.

In summary, the above proposed patient referral policy, QoS approximation methods, and optimization algorithm combined together form an effective and computationally efficient new framework for achieving much better service for all patients while meeting the QoS requirements for each individual patient type in an ICU network.

### 9 Hardware

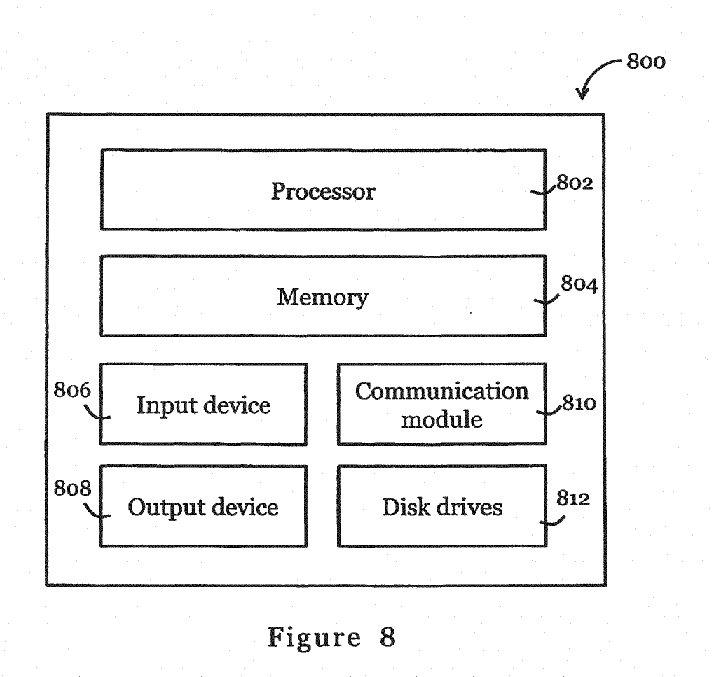

FIG. 8 shows a schematic diagram of an exemplary information handling system 800 that can be used as an information handling system in one embodiment of the invention for enabling operation of the methods in the above embodiments. The information handling system 800 may have different configurations, and it generally comprises suitable components necessary to receive, store and execute appropriate computer instructions or codes. The main components of the information handling system 800 are a processing unit 802 and a memory unit 804. The processing unit 802 is a processor such as a CPU, an MCU, etc. The memory unit 804 may include a volatile memory unit (such as RAM), a non-volatile unit (such as ROM, EPROM, EEPROM and flash memory) or both. Preferably, the information handling system 800 further includes one or more input devices 806 such as a keyboard, a mouse, a stylus, a microphone, a tactile input device (e.g., touch sensitive screen) and a video input device (e.g., camera). The information handling system 800 may further include one or more output devices 808 such as one or more displays, speakers, disk drives, and printers. The displays may be a liquid crystal display, a light emitting diode display or any other suitable display that may or may not be touch sensitive. The information handling system 800 may further include one or more disk drives 812 which may encompass solid state drives, hard disk drives, optical drives and/or magnetic tape drives. A suitable operating system may be installed in the information handling system 800, e.g., on the disk drive 812 or in the memory unit 804 of the information handling system 800. The memory unit 804 and the disk drive 812 may be operated by the processing unit 802. The information handling system 800 also preferably includes a communication module 810 for establishing one or more communication links (not shown) with one or more other computing devices such as a server, personal computers, terminals, wireless or handheld computing devices. The communication module 810 may be a modem, a Network Interface Card (NIC), an integrated network interface, a radio frequency transceiver, an optical port, an infrared port, a USB connection, or other interfaces. The communication links may be wired or wireless for communicating commands, instructions, information and/or data. Preferably, the processing unit 802, the memory unit 804, and optionally the input devices 806, the output devices 808, the communication module 810 and the disk drives 812 are connected with each other through a bus, a Peripheral Component Interconnect (PCI) such as PCI Express, a Universal Serial Bus (USB), and/or an optical bus structure. In one embodiment, some of these components may be connected through a network such as the Internet or a cloud computing network. A person skilled in the art would appreciate that the information handling system 800 shown in FIG. 2 is merely exemplary, and that different information handling systems 800 may have different configurations and still be applicable in the invention.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the invention are either wholly implemented by computing systems or partly implemented by computing systems then any appropriate computing system architecture may be utilized. This will include stand-alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for managing admission of patients into one of a plurality of intensive care units in a hospital network, comprising:
classifying a patient arriving at a first intensive care unit of the plurality of intensive care units as one of an external emergency patient, an internal emergency patient, and an elective patient; and
determining whether to admit the patient to the intensive care units in the hospital network based on the classification so as to
admit an external emergency patient to the first intensive care unit if a vacancy in the first intensive care unit is above a first threshold;
admit an internal emergency patient to the first intensive care unit if the first intensive care unit has vacancy; and
admit an elective patient to the first intensive care unit if a vacancy in the first intensive care unit is above a second threshold;
wherein the first and second thresholds are independent of the classification of the patient.

2. The method of claim 1, further comprising rejecting admission of the elective patient to the intensive care units in the hospital network if the vacancy at the first intensive care unit falls below the second threshold.

3. The method of claim 1, further comprising rejecting admission of the external emergency patient to the first intensive care unit if a vacancy in the first intensive care unit is below a first threshold; and determining whether to admit the external emergency patient to another intensive care unit in the hospital network.

4. The method of claim 3, wherein the determination of whether to admit the external emergency patient to another intensive care unit in the hospital network is based on an overflow control method.

5. The method of claim 4, wherein the overflow control method comprising repeating the determination step for each of the other intensive care units in a hospital network.

6. The method of claim 5, further comprising admitting the external emergency patient to another intensive care unit in the hospital network if a vacancy in the respective intensive care unit is above a respective third threshold.

7. The method of claim 6, further comprising rejecting admission of the external emergency patient to the intensive care units in the hospital network if vacancies at all of the respective intensive care units are below the first threshold or the respective third threshold.

8. The method of claim 6, wherein the first and third thresholds are equal.

9. The method of claim 6, wherein the first and third thresholds are adjustable dynamically.

10. The method of claim 6, wherein the first and third thresholds are fixed.

11. The method of claim 1, wherein first and second thresholds are adjustable dynamically.

12. The method of claim 1, wherein the first and second thresholds are fixed.

13. The method of claim 1, further comprising evaluating a first quality of service for internal emergency patients and a second quality of service for elective patients based on exponential decomposition method with moment matching

14. The method of claim 1, further comprising evaluating a third quality of service for external emergency patients based on information exchange surrogate approximation method.

15. The method of claim 6, further comprising evaluating a first quality of service for internal emergency patients and a second quality of service for elective patients based on exponential decomposition method with moment matching; evaluating a third quality of service for external emergency patient based on information exchange surrogate approximation method; and determining the first, second, and third thresholds based on the first, second, and third evaluated quality of service.

16. A system for managing admission of patient into one of a plurality of intensive care units in a hospital network, comprising:
means for classifying a patient arriving at a first intensive care unit of the plurality of intensive care units as one of an external emergency patient, an internal emergency patient, and an elective patient; and
means for determining whether to admit the patient to the intensive care units in the hospital network based on the classification so as to
admit an external emergency patient to the first intensive care unit if a vacancy in the first intensive care unit is above a first threshold;
admit an internal emergency patient to the first intensive care unit if the first intensive care unit has vacancy; and
admit an elective patient to the first intensive care unit if a vacancy in the first intensive care unit is above a second threshold;
wherein the first and second thresholds are independent of the classification of the patient.

17. A non-transitory computer readable medium for storing computer instructions that, when executed by one or more processors, causes the one or more processors to perform method for managing admission of patient into one of a plurality of intensive care units in a hospital network, comprising:
classifying a patient arriving at a first intensive care unit of the plurality of intensive care units as one of an external emergency patient, an internal emergency patient, and an elective patient; and
determining whether to admit the patient to the intensive care units in the hospital network based on the classification so as to
admit an external emergency patient to the first intensive care unit if a vacancy in the first intensive care unit is above a first threshold;
admit an internal emergency patient to the first intensive care unit if the first intensive care unit has vacancy; and
admit an elective patient to the first intensive care unit if a vacancy in the first intensive care unit is above a second threshold;
wherein the first and second thresholds are independent of the classification of the patient.
